(19) United States
(12) Patent Application Publication
(10) Pub. No.: US 2006/0205807 A1
(43) Pub. Date: Sep. 14, 2006

(54) Amino-chroman compounds and methods for preparing same

(75) Inventors: Christoph Dehnhardt, New York, NY (US); Sylvain Daigneault, Laval (CA)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/355,409

(22) Filed: Feb. 16, 2006

Related U.S. Application Data
(60) Provisional application No. 60/653,327, filed on Feb. 16, 2005.

(51) Int. Cl. A61K 31/353; C07D 311/04

(52) U.S. Cl. 514/456; 549/403

(57) ABSTRACT

The present invention relates to novel amino-chroman compounds, methods of preparing such compounds, and to synthetic intermediates useful in such methods.

### CROSS REFERENCE TO RELATED APPLICATIONS

The present application claims benefit of Provisional Application Ser. No. 60/653,327, filed Feb. 16, 2005, the disclosure of which is hereby incorporated by reference in its entirety.

### FIELD OF THE INVENTION

The present invention relates to novel amino-chroman compounds, to methods of preparing amino-chroman compounds, and to synthetic intermediates useful in such methods.

### BACKGROUND OF THE INVENTION

Major depressive disorder affects an estimated 340 million people worldwide. Depression is the most frequently diagnosed psychiatric disorder and, according to the World Health Organization, is the fourth greatest public health problem. If left untreated, the effects of depression can be devastating, robbing people of the energy or motivation to perform everyday activities and, in some cases, leading to suicide. Symptoms of the disorder include feelings of sadness or emptiness, lack of interest or pleasure in nearly all activities, and feelings of worthlessness or inappropriate guilt. In addition to the personal costs of depression, the disorder also has been estimated to result in more than $40 billion in annual costs in the United States alone, due to premature death, lost productivity, and absenteeism.

Selective serotonin reuptake inhibitors (SSRIs) have had significant success in treating depression and related illnesses and have become among the most prescribed drugs since the 1980s. Some of the most widely known SSRIs are fluoxetine, sertraline, paroxetine, fluvoxamine and citalopram. Although they have a favorable side effect profile compared to tricyclic antidepressants (TCAs), they have their own particular set of side effects due to the non-selective stimulation of serotonergic sites. They typically have a slow onset of action, often taking several weeks to produce their full therapeutic effect. Furthermore, they have generally been found to be effective in less than two-thirds of patients.

SSRIs are believed to work by blocking the neuronal reuptake of serotonin, increasing the concentration of serotonin in the synaptic space, and thus increasing the activation of postsynaptic serotonin receptors. Although a single dose of a SSRI can inhibit the neuronal serotonin transporter, and thus would be expected to increase synaptic serotonin, clinical improvement has generally been observed only after long-term treatment. It has been suggested that the delay in onset of antidepressant action of the SSRIs is the result of an increase in serotonin levels in the vicinity of the serotonergic cell bodies. This excess serotonin is believed to activate somatodendritic autoreceptors, i.e., 5-HT_{1A} receptors, reduce cell firing activity and, in turn, decrease serotonin release in major forebrain areas. This negative feedback limits the increment of synaptic serotonin that can be induced by antidepressants acutely. Over time, the somatodendritic autoreceptors become desensitized, allowing the full effect of the SSRIs to be expressed in the forebrain. This time period has been found to correspond to the latency for the onset of antidepressant activity [Perez, V., et al., The Lancet, 1997, 349: 1594-1597].

In contrast to the SSRIs, a 5-HT_{1A} agonist or partial agonist acts directly on postsynaptic serotonin receptors to increase serotonergic neurotransmission during the latency period for the SSRI effect. Accordingly, the 5-HT_{1A} partial agonists buspirone and gepirone [Feiger, A., Psychopharmacol. Bull., 1996, 32(4): 659-665; Wilcox, C., Psychopharmacol. Bull., 1996, 32(93): 335-342], and the 5-HT_{1A} agonist flesinoxan [Grof, P., International Clinical Psychopharmacology, 1993, 8(3): 167-172], have shown efficacy in clinical trials for the treatment of depression. Furthermore, such agents are believed to stimulate the somatodendritic autoreceptors, thus hastening their desensitization and decreasing the SSRI latency period. An agent with a dual mechanism of antidepressant action would be expected to have greater efficacy and thus reduce the number of patients refractory to treatment. Indeed, buspirone augmentation to standard SSRI therapy has been shown to produce marked clinical improvement in patients initially unresponsive to standard antidepressant therapy [Dimitriou, E., J. Clinical Psychopharmacol., 1998, 18(6): 465-469].

Concurrently filed application Ser. No. 10/898,866, the disclosure of which is hereby incorporated by reference in its entirety, teaches 3-amino chroman compounds that are useful, for example, as serotonin reuptake inhibitors, and as 5-HT_{1A} receptor agonists or antagonists. Additional amino chroman compounds, and alternative procedures for synthesizing these and other amino-chroman compounds would be desirable.

### SUMMARY OF THE INVENTION

In one embodiment, the present invention provides amino-chroman compounds of formula I:

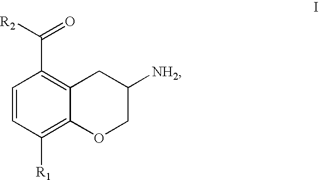

wherein R₁ is H or halogen and R₂ is a group capable of bearing a net negative charge. In preferred compounds of this type, R₂ is OR₃, heteroaryl, SR₄, or NR₅R₆, wherein:

R₃ and R₄ are independently, alkyl, aryl, or heteroaryl; and

R₅ and R₆ are independently, H, alkyl, aryl, or OR₃.

Preferably, R₁ is F and R₂ is OR₃, where R₃ is C₁-C₁₂ alkyl. Compounds in which R₂ is methoxy are particularly preferred, including 3-amino-8-fluoro-chroman-5-carboxylic acid methyl ester.

Compounds of formula I preferably are prepared by hydrogenating compounds of formula II: 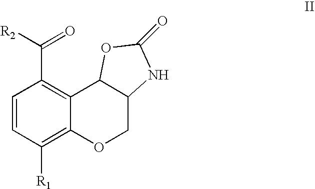
in a solvent. In one embodiment, the first solvent is a polar solvent, such as acetic acid, tetrahydrofuran (THF), and the like. Preferably, the first solvent is a polar organic solvent such as a C₁-C₅ alcohol, ether, cyclic ether, water, alkyl acetate, acetic acid, or a mixture thereof, preferably in the presence of a catalyst.

### DETAILED DESCRIPTION OF ILLUSTRATIVE EMBODIMENTS

In accordance with the present invention, a group that is capable of bearing a net negative charge is one that exhibits a level of stability in a solvent of choice sufficient to facilitate its displacement following nucleophillic attack on the carbonyl group to which it is attached. Any of the many known groups of this type can be employed including, for example, alkoxy groups, aryloxy groups, heteroaryl groups, thio groups, or amino groups. Use of an alkoxy group, particularly a methoxy group, is preferred.

The term “alkyl”, as used herein, whether used alone or as part of another group, refers to an aliphatic hydrocarbon chain and includes, but is not limited to, straight and branched chains having 1 to 6 carbon atoms unless otherwise specified. For example, methyl, ethyl, n-propyl, isopropyl, and 2-methylpropyl are encompassed by the term “alkyl”. Specifically included within the definition of “alkyl” are those aliphatic hydrocarbon chains that are optionally substituted, including unsubstituted and mono-, di- and tri-substituted hydrocarbon groups. Suitable substitutions include, for example, halogen substituents and the like.

Carbon number, as used in the definitions herein, refers to carbon backbone and carbon branching, but does not include carbon atoms of substituents, such as alkoxy substitutions and the like.

The term “aryl” means an aromatic carbocyclic moiety of up to 20 carbon atoms, which may be a single ring (monocyclic) or multiple rings (bicyclic, up to three rings) fused together or linked covalently. Any suitable ring position of the aryl moiety may be covalently linked to the defined chemical structure. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, acenaphthylenyl, and the like. The term “aryl” further includes both unsubstituted carbocylic groups and carbocylic groups containing 1-5-substitutions.

The term “heteroaryl” as used herein means an aromatic heterocyclic ring system, which may be a single ring (monocyclic) or multiple rings (bicyclic, up to three rings) fused together or linked covalently. The rings may contain from one to four hetero atoms selected from nitrogen, oxygen, or sulfur, wherein the nitrogen or sulfur atom(s) are optionally oxidized, or the nitrogen atom(s) are optionally quarternized. Any suitable ring position of the heteroaryl moiety may be covalently linked to the defined chemical structure. Examples of heteroaryl moieties include, but are not limited to, heterocycles such as furan, thiophene, pyrrole, N-methylpyrrole, pyrazole, N-methylpyrazole, imidazole, N-methylimidazole, oxazole, isoxazole, thiazole, isothiazole, 1H-tetrazole, 1-methyltetrazole, 1,3,4-oxadiazole, 1H-1,2,4-triazole, 1-methyl-1,2,4-triazole 1,3,4-triazole, 1-methyl-1,3,4-triazole, pyridine, pyrimidine, pyrazine, pyridazine, benzoxazole, benzisoxazole, benzothiazole, benzofuran, benzothiophene, thianthrene, dibenzo[b,d]furan, dibenzo[b,d]thiophene, benzimidazole, N-methylbenzimidazole, indole, indazole, quinoline, isoquinoline, quinazoline, quinoxaline, purine, pteridine, 9H-carbazole, β-carboline, and the like.

Heteroaryl chemical groups, as herein before defined, are also optionally saturated or partial saturated heterocyclic rings. Examples of saturated or partially saturated heteroaryl moieties include, but are not limited to, chemical groups such as azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, tetrahydroisoquinolinyl, and the like.

The term “halogen” designates fluorine, chlorine, iodine, and bromine.

Polar solvents are those that at least partially dissolve the starting material and product. Representative solvents of this type include C₁-C₆ alcohols, acetic acid, THF, ether, and the like, and mixtures thereof. C₁-C₆ alcohols, ether, cyclic ether, water, alkyl acetate, and acetic acid are preferred, particularly methanol and ethanol.

Any of the many known hydrogenation catalysts known in the art can be used in the instant methods, including, for example, palladium on carbon (Pd/C), palladium (black), platinum, Raney nickel, and rhodium on carbon (Rh/C).

The hydrogenation reaction is carried out preferably at room temperature, preferably under 1 atm hydrogen pressure. Hydrogen gas can be replaced with other hydrogen sources, for example, cyclohexadiene, formic acid, and the like.

Compounds of formula II preferably are prepared by reacting a salt of formula III: 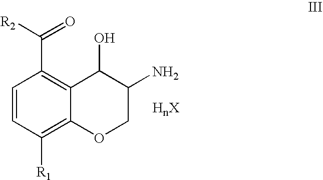
wherein n is an integer of 1 or 2 and X is a halogen, sulfate, phosphate, alkylsulfonate, or carboxylate, with carbonyldiimidazole, phosgene, or alkylchloroformiate, in the presence of an organic or inorganic base such as triethylamine or sodium bicarbonate. Preferably, n is 1 and X is Cl. This reaction preferably is performed in a solvent, such as a polar aprotic solvent, a polar protic solvent, or a non-polar aprotic solvent. Preferably, the solvent is CH₃CN, ethyl acetate, chloroform or a mixture thereof.

Salts of formula III, in turn, preferably are prepared by hydrogenating a compound of formula IV: 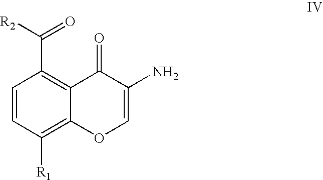
in the presence of an acid, in a polar solvent that is capable of dissolving compounds of formulas III and IV, such as a C₁-C₆ alcohol, acetic acid, THF, ether, cyclic ether, water, alkyl acetate, or mixtures thereof. Representative acids include strong acids such as HCl, H₂SO₄, HClO₄, formic acid, phosphoric acid, acetic acid, and trifluoroacetic acid. C₁-C₆ alcohols are preferred, particularly methanol and ethanol. This hydrogenation reaction also should be performed in the presence of one of the above-noted catalysts.

Compounds of formula IV can be prepared by contacting a compound of formula V: 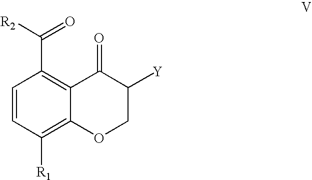
wherein Y is a halogen, arylsulfonate or alkysulfonate, with NaN₃ in DMF or some other polar aprotic solvent. Preferably, Y is Br or Cl.

Compounds of formula V can be prepared by contacting a compound of formula VI: 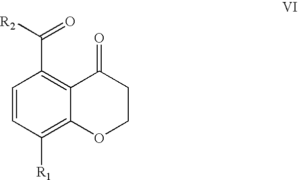
with a halogenating agent, such as Cl₂ or Br₂, or a metal halide, preferably a Group Ib metal halide such as, for example, CuZ₂ ,PZ₃, Z₂, PZ₅ where Z is Br or Cl. Preferred halogenating agents include CuBr₂. Compounds of formula VI can be made by cyclizing a compound of formula VII: 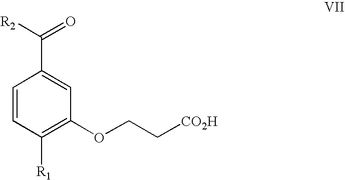
through reaction with a chlorinating agent such as PCl₅, SOCl₂, oxalylchloride, and the like, followed by reaction with a Lewis acid, such as AlCl₃, SnCl₄, TiCl₄, or BF₃. Alternatively, direct cyclization can be accomplished under strongly acidic and dehydrating conditions with polyphosphoric acid or Eatons reagent. Compounds of formula VII, in turn, can be made by contacting a compound of formula VIII: 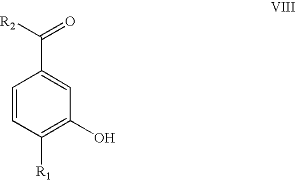
with β-propioactone in the presence of an organic or inorganic base, such alkali t-butoxide, -hydrides, -hydroxides, -carbonates, or akloxides. This reaction preferably is performed in a solvent such as THF, DMF, dioxan, water, C₁-C₆ alcohols, or mixtures thereof.

The present invention is also directed to the compounds employed in the foregoing methods. Preferred compounds of formula II include 6-fluoro-2-oxo-2,3,3a,9b-tetrahydro-4H-chromeno[3,4-d]oxazole-9-carboxylic acid methyl ester, preferred compounds of formula III include 3-amino-8-fluoro-4-hydroxy-chroman-5-carboxylic acid methyl ester-hydrochloride, preferred compounds of formula IV include 3-amino-8-fluoro-4-oxo-4H-chromene-5-carboxylic acid methyl ester, preferred compounds of formula V include 3-bromo-8-fluoro-4-oxo-chroman-5-carboxylic acid methyl ester, preferred compounds of formula VI include 8-fluoro-4-oxo-chroman-5-carboxylic acid methyl ester, and preferred compounds of formula VII include 3-(2-carboxy-ethoxy)-4-fluoro-benzoic acid methyl ester.

In one embodiment, the present invention is directed to compounds of formula VIII, including 4-fluoro-3-hydroxy-benzoic acid methyl ester.

Tautomers often exist in equilibrium with each other. As these tautomers interconvert under environmental and physiological conditions, they provide the same useful biological effects. The present invention encompasses mixtures of such tautomers.

The compounds of this invention may contain an asymmetric carbon atom and some of the compounds of this invention may contain one or more asymmetric centers and may thus give rise to optical isomers and diastereomers. While shown without respect to stereochemistry in formula II, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one steriosomer, preferably less than about 50%, more preferably less than about 75%, and even more preferably less than about 90%.

The compounds of the present invention can be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived from organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

Methods of Making

Compounds of formula I can be synthesized generally according to the procedure of Scheme 1. Briefly, ester 5 deprotonates with KOt-Bu and reacts with β-butylrolactone to form β-phenoxypropionic acid 11 in 90% yield following a literature protocol. See Buckle, D. R., Eggleston, D. S., et al., J. Chem. Soc. Perkin Trans. I., 1991, 2763. Acid 11 may be cyclized to compound 12, via acid chloride and ring closure with AlCl₃ or TFOH, or by direct cyclization of 11 using Eaton's reagent. The latter provides lower yields and less pure material. Ketone 12 can be brominated with CuBr₂ in 61% yield. Introduction of nitrogen may be achieved with NaN₃ to give aminoketone 14 in yields between 80% and 90%.

Compared to the literature, [see Patonay, T. et al., Liebigs Ann. Chem., 1979, 162; Litkei, G., et al., Liebigs Ann. Chem., 1979, 174], intermediary formed azide is not isolable because it hydrolyses under base catalysis forming 14. Catalytic hydrogenation leads to the desired amino hydroxychromanol, which crystallizes from 2-propanol/water as HCl salt 15. Salt 15 can be converted to the cyclic carbamate 16 using CDI in CH₃CN. The hydrogenolysis of the benzylic oxy-group works quantitatively with Pd/C in ethanol to yield formula I, where R₁ is F and R₂ is methoxy. The overall synthesis has a yield of about 17%.

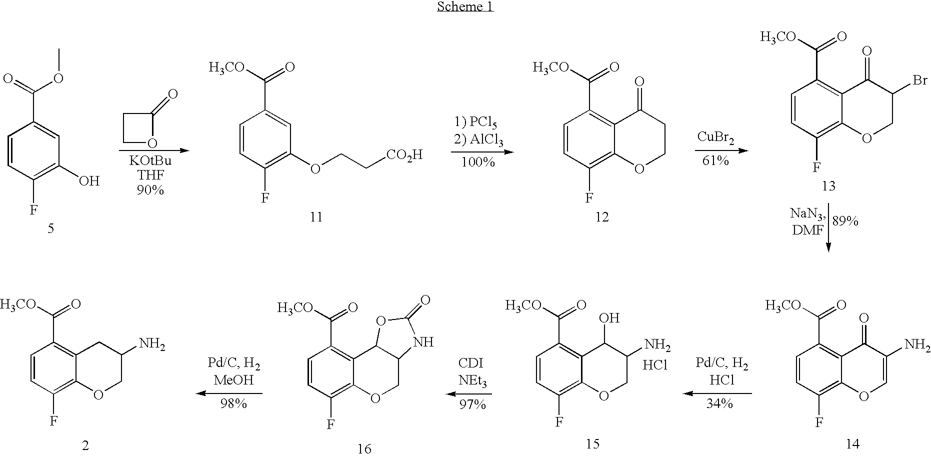

The present compounds are further described in the following examples. The following abbreviations are used: DMF is N,N-dimethylformamide, CDI is carbonyldiimidazole, THF is tetrahydrofuran, DMSO is dimethyl sulfoxide, NMR is proton nuclear magnetic resonance, and MS is mass spectroscopy with (+) referring to the positive mode which generally gives a M+1 (or M+H) absorption where M=the molecular mass. Unless otherwise noted, all compounds are analyzed at least by MS and NMR.

### EXAMPLES

### Example 1

### Synthesis of 3-(2-carboxy-ethoxy)-4-fluoro-benzoic acid methyl ester

4-Fluoro-3-hydroxy-benzoic acid methyl ester (14.4g, 84.55 mmol) was suspended in THF (100 mL) and cooled to 0-5° C. KOt-Bu (9.94 g, 88.77 mmol, 1.05 eq) dissolved in THF (50 mL) was added over 10 minutes. β-propiolactone (7.45g, 93.01 mmol, 1.1 eq) was added in one portion. The reaction mixture was warmed to RT for 1 hr and then to 50° C. for 3 hrs. After reaction completion, 10 mL sat. NaHCO₃ and water (90 mL) were added to the suspension. EtOAc (90 mL) were added and the aqueous layer was separated. 15% HCl (25 mL) was added to the solution, so that pH=0-2. The aqueous layer was extracted with EtOAc (3×50 mL). The combined organic layers were dried over MgSO₄. After solvent removal, 18.45 g (90%) of the title compound was obtained as white solid. ¹H-NMR (300 MHz, D₆-DMSO); δ=2.73 (t, j=6.0 Hz, 2H), 3.86 (s, 3H), 4.31 (t, j=6.0 Hz, 2H), 7.35 (dd, j=8.3, 11.1 Hz, 1H), 7.57-7.62 (m, 1H), 7.66 (dd, j=2.0, 8.3 Hz, 1H), 12.45 (s, 1H) ppm.

### Example 2

### Synthesis of 8-fluoro-4-oxo-chroman-5-carboxylic acid methyl ester

3-(2-Carboxy-ethoxy)-4-fluoro-benzoic acid methyl ester (15.0g, 61.93 mmol) was suspended in CH₂Cl₂ (50 mL) and PCl₅ (12.9 g, 61.93 mmol) was added. The solution was stirred for 1 hr at RT and then added dropwise to a suspension of AlCl₃ (25.97g. 194.8 mmol, 3.15 eq) in CH₂Cl₂ (50 mL) at RT. The reaction mixture was stirred for 1 hr and was poured onto ice/water (250 mL) and stirred for 5 min. The layers were separated and the organic layer was extracted with CH₂Cl₂ (100 mL). The combined organic layers were treated with solid NaHCO₃ (5 g) and water (20 mL). A cloudy solution was obtained after decantation, which was treated with brine (50 mL) and dried over MgSO₄. The solvent was removed under reduced pressure to give the title compound (14.0 g, 100%) as oil, which solidified upon standing. ¹H-NMR (300 MHz, D₆-DMSO): δ=2.90 (t, j=6.4 Hz, 2H), 3.79 (s, 3H), 4.68 (t, j=6.4 Hz,2H), 7.04 (dd, j=4.4, 8.4 Hz, 1H), 7.58 (dd, j=10.7, 8.4 Hz, 1H) ppm.

### Example 3

### Synthesis of 3-bromo-8-fluoro-4-oxo-chroman-5-carboxylic acid methyl ester

8-Fluoro-4-oxo-chroman-5-carboxylic acid methyl ester (70 g, 31.11 mmol) was dissolved in CHCl₃ (35 mL) and added to a slurry of CuBr₂ (12.5 g, 55.99 mmol, 1.8 eq) in EtOAc (35 mL). The reaction mixture was heated to reflux at 75° C. for 3 hr. The suspension was filtered over celite and the filtrate was distilled in vacuum to constant weight. Heptane/EtOAc (3:1) (20 mL) was added to the distillate and the white solid was collected by filtration giving 4.7 g of the title product. The mother liqueur was distilled and the residue purified by column chromatography with heptane/EtOAc yielding another 1.09 g of the title product so that the overall yield for this step was 5.79 g (61%). ¹H-NMR (300 MHz, D6-DMSO): δ=3.81 (s, 3H), 4.78 (dd, j=4.0, 13.3 Hz, 1H), 5.03 (dd, j=2.7, 13.3 Hz, 1H), 5.13 (dd, j=2.7,4.0 Hz, 1H), 7.15 (dd, j=4.5, 8.4 Hz, 1H), 7.69 (dd, j=8.4, 10.5 Hz, 1H) ppm.

### Example 3

### Synthesis of 3-amino-8-fluoro-4-oxo-4H-chromene-5-carboxylic acid methyl ester

3-Bromo-8-fluoro-4-oxo-chroman-5-carboxylic acid methyl ester (6.49 g, 21.42 mmol) was dissolved in DMF (65 mL) and cooled to 0-1° C. NaN₃ (1.83 g, 28.12 mmol, 1.31 eq) was added in 5 portions over 3 hr and the mixture was allowed to stir for additional 3 hr at 5-10° C. The dark solution was poured onto water (250 mL) and was extracted with EtOAc (2×100 mL). The remaining aqueous layer was basified with 1N NaOH and again extracted with EtOAc (50 mL). The combined organic layers were washed with water (50 mL) and brine (50 mL) and dried over MgSO₄. Solvents were removed on a rotavapor in vacuum with bath temperature less than 30° C. to obtain 4.52 g (89%) of a brownish solid title compound. ¹H-NMR (300 MHz, D₆-DMSO): δ=3.84 (s, 3H), 4.77 (s, 2H), 7.36 (dd, j=4.5, 8.2 Hz, 1H), 7.71 (dd, j=8.2, 10.7 Hz,m 1H), 8.06 (s, 1H) ppm.

### Example 4

### Synthesis of 3-amino-8-fluoro-4-hydroxy-chroman-5-carboxylic acid methyl ester-hydrochloride

3-Amino-8-fluoro-4-oxo-4H-chromene-5-carboxylic acid methyl ester (82% pure) (1.50 g, 633 mmol) was dissolved in MeOH (60 mL); HCl (28% in MeOH) (2 mL) and Pd/C (10%, 50% wet) (800 mg) were added. The mixture was hydrogenated at 48 psi over 26 hr. The reaction mixture was filtered over celite and the filtrate was distilled under reduced pressure to obtain 11.54 g of a red solid. The crude material was recrystalized in 13 mL isopropanol and 1 mL water to obtain the title compound (600 mg, 34%). ¹H-NMR (300 MHz, D₆-DMSO): δ=3.71 (m, 1H), 3.84 (s, 3H), 4.23 (t, j=10.3 Hz, 1H), 4.39 (dd, j=3.2, 10.3 Hz, 1H), 5,52 (t, j=4.8Hz, 1H), 6.15 (d, j=5.7 Hz, 1H), 7.34 (dd, j=8.6, 10.4 Hz, 1H), 7.44 (dd, j=5.1, 8.6 Hz, 1H), 8.37 (s, 3H) ppm.

### Example 5

### Synthesis of 6-fluoro-2-oxo-2,3,3a,9b-tetrahydro-4H-chromeno[3,4-d]oxazole-9-carboxylic acid methyl ester

3-Amino-8-fluoro-4-hydroxy-chroman-5-carboxylic acid methyl ester-hydrochloride (300 mg, 1.08 mmol) was suspended in CH₃CN (3 mL) and NEt₃ (220 mg, 2.16 mmol) was added. The mixture was stirred for 30 min and CDI (225 mg, 1.39 mmol, 1.29 eq) was added and the mixture was allowed to stir for another 4 hr. After complete reaction, CH₃CN was removed in vacuum and the residue was dissolved in EtOAc/MeOH (9:1) and filtered through filter funnel with 1 g silicagel. The filtrate was distilled in vacuum to yield the title compound (280 mg, 97%) as white solid. ¹H-NMR (300 MHz, D₆-DMSO): δ=3.85 (s, 3H), 4.17 (m, 2H), 4.32 (m, 1H), 6.35 (d, j=8.6 Hz, 1H), 7.41 (dd, j=8.6, 10.2 Hz, 1H), 7.57 (dd, j=5.1, 8.6 Hz, 1H), 8.02 (s, 1H) ppm.

### Example 6

### Synthesis of 3-amino-8-fluoro-chroman-5-carboxylic acid methyl ester

6-Fluoro-2-oxo-2,3,3a,9b-tetrahydro-4H-chromeno[3,4-d]oxazo 1 e-9-carboxylic acid methyl ester (240 mg, 0.90 mmol) was dissolved in EtOH/MeOH (1:2) (15 mL) and Pd/C (5%) (50 mg) was added. The mixture was hydrogenated over night at atmospheric pressure. The catalyst was removed by filtration over celite and the solvent was distilled in vacuum to yield 200 mg (98%) of the title compound as white solid. ¹H-NMR (300 MHz, D₆-DMSO): δ=2.72 (dd, j=7.7, 17.1 Hz, 1H), 3.25-3.09 (m, 2H), 3.71 (dd, j=8.0, 10.1 Hz, 1H), 3.81 (s, 3H), 4.17 (m, 1H), 7.16 (dd, j=8.7, 10.4 Hz, 1H), 7.42, (dd, j=5.2, 8.7 Hz, 1H) ppm.

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

What is claimed:

1. A compound of formula I: 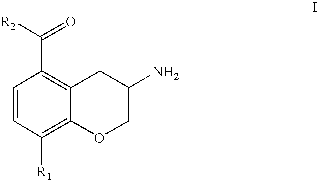
wherein R₁ is H or a halogen and R₂ is a group capable of bearing an net negative charge.

2. The compound of claim 1, wherein R₁ is F and R₂ is OR₃, heteroaryl, SR₄, or NR₅R₆, wherein:
R₃ and R₄ are independently, alkyl, aryl, or heteroaryl; and
R₅ and R₆ are independently, H, alkyl, aryl, or OR₃.

3. The compound of claim 1, wherein R₁ is F and R₂ is OR₃.

4. The compound of claim 1, wherein R₁ is F and R₂ is methoxy.

5. The compound of claim 1, wherein the compound of formula I is 3-amino-8-fluoro-chroman-5-carboxylic acid methyl ester.

6. A method for preparing compounds of formula I: 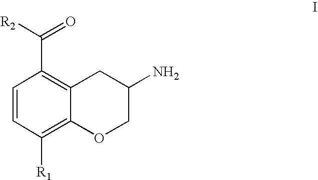
wherein R₁ is H or a halogen and R₂ is a group capable of bearing an net negative charge, comprising:
hydrogenating a compound of formula II: 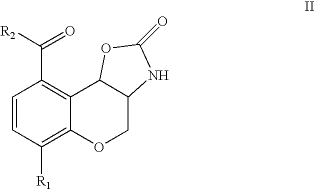
in a solvent.

7. The method of claim 6, wherein R₂ is OR₃ and R₃ is alkyl, aryl, or heteroaryl.

8. The method of claim 7, wherein R₃ is C₁-C₁₂ alkyl.

9. The method of claim 6, wherein the solvent is a C₁-C₅ alcohol, ether, cyclic ether, water, alkyl acetate, acetic acid, THF, or mixtures thereof.

10. The method of claim 6, wherein the first solvent is methanol or ethanol.

11. The method of claim 6, wherein the method further includes a catalyst.

12. The method of claim 1 1, wherein the catalyst is palladium on carbon, palladium (black), platinum, Raney nickel, or rhodium on carbon.

13. The method of claim 6, wherein the compound of formula II is 6-fluoro-2-oxo-2,3,3a,9b-tetrahydro-4H-chromeno[3,4-d]oxazole-9-carboxylic acid methyl ester.

14. A compound of formula I prepared according to the method of claim 6.

15. The method of claim 6, wherein compounds of formula II are prepared by reacting a salt of formula III: 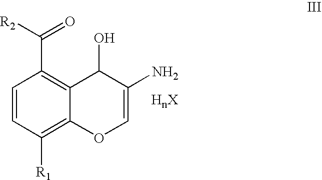
wherein n is an integer of 1 or 2, X is a halogen, sulfate, phosphate, alkylsufonate, or carboxylate, with carbonyldiimidazole, phosgene, or alkylchloroformiate, in the presence of a base.

16. The method of claim 15, wherein the base is triethylamine or sodium bicarbonate.

17. The method of claim 15, wherein the method further includes a second solvent.

18. The method of claim 17, wherein the second solvent is CH₃CN, ethyl acetate, choloform, or mixtures thereof.

19. The method of claim 15, wherein the compound of formula III is 3-amino-8-fluoro-4-hydroxy-chroman-5-carboxylic acid methyl ester-hydrochloride.

20. A compound of formula II prepared according to the method of claim 15.

21. The method of claim 15, wherein the salts of formula III are prepared by hydrogenating a compound of formula IV: 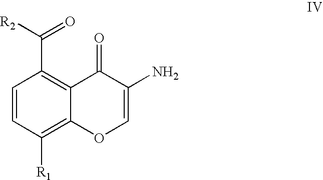
in the presence of an acid.

22. The method of claim 21, wherein the acid is HCl, H₂SO₄, HClO₄, formic acid, phosphoric acid, acetic acid, trifluoroacetic acid, or mixtures thereof.

23. The method of claim 21, wherein the method further includes a polar solvent.

24. The method of claim 23, wherein the polar solvent is a C₁-C₁₂ alcohol, acetic acid, THF, ether, cyclic ether, water, alkyl acetate, or mixtures thereof.

25. The method of claim 23, wherein the polar solvent is methanol or ethanol.

26. The method of claim 21, wherein the method further includes a catalyst.

27. The method of claim 26, wherein the catalyst is palladium on carbon, platinum, palladium (black), Raney nickel, or rhodium on carbon.

28. The method of claim 21, wherein the compound of formula IV is 3-amino-8-fluoro-4-oxo-4H-chromene-5-carboxylic acid methyl ester.

29. A compound of formula III prepared according to the method of claim 21;

30. The method of claim 21, wherein compounds of formula IV are prepared by contacting a compound of formula V: 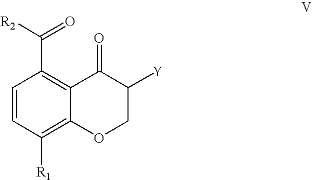
wherein Y is halogen, arylsulfonate, or alkylsulfonate, with NaN₃ in a polar aprotic solvent.

31. The method of claim 30, wherein Y is Br or Cl.

32. The method of claim 30, wherein the polar aprotic solvent is DMF.

33. The method of claim 30, wherein the compound of formula V is 3-bromo-8-fluoro-4-oxo-chroman-5-carboxylic acid methyl ester.

34. A compound of formula IV prepared according to the method of claim 30.

35. The method of claim 27, wherein compounds of formula V are prepared by contacting a compound of formula VI: 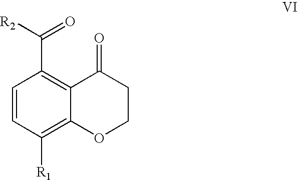
with a halogenating agent or a metal halide.

36. The method of claim 35, wherein the metal halide is a Group Ib metal halide.

37. The method of claim 35, wherein the metal halide is CuZ₂, PZ₂, Z₂, or PZ₅, where Z is Br or Cl.

38. The method of claim 35, wherein the compound of formula VI is 8-fluoro-4-oxo-chroman-5-carboxylic acid methyl ester.

39. A compound d of formula V prepared according to the method of claim 35.

40. The method of claim 35, wherein compounds of formula VI are prepared by cyclizing a compound of formula VII: 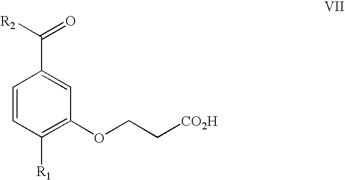
through reaction with a chlorinating agent and a Lewis acid.

41. The method of claim 40, wherein the chlorinating agent is PCl₅, SOCl₂, oxalychloride, or mixtures thereof.

42. The method of claim 40, wherein the Lewis acid is AlCl₃, SnCl₄, TiCl₄, BF₃, or mixtures thereof.

43. The method of claim 40, wherein the compound of formula VII is 3-(2-carboxy-ethoxy)-4-fluoro-benzoic acid methyl ester.

44. A compound of formula VI prepared according to the method of claim 40.

45. The method of claim 40, wherein compounds of formula VII are prepared by contacting a compound of formula VIII: 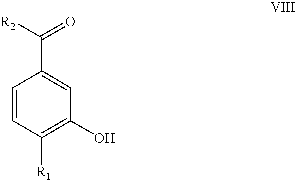
with β-propioactone in the presence of a base.

46. The method of claim 45, wherein the base is alkali t-butoxide, alkali-hydride, alkali-hydroxide, alkali-carbonate, or alkoxide.

47. The method of claim 45, wherein the method further includes a polar or non-polar solvent.

48. The method of claim 47, wherein the polar or non-polar solvent in THF, DMF, dioxin, water, C₁-C₆ alcohol, or mixtures thereof.

49. The method of claim 45, wherein the compound of formula VIII is 4-fluoro-3-hydroxy-benzoic acid methyl ester.

50. A compound of formula VII prepared according to the method of claim 45.
